(19) United States
(12) Patent Application Publication
(10) Pub. No.: US 2015/0121058 A1
(43) Pub. Date: Apr. 30, 2015

(54) Intelligent Real-time Optimization

(71) Applicants: Alexander Gebhart, Walldorf (DE); Clemens Daeschle, Walldorf (DE)

(72) Inventors: Alexander Gebhart, Walldorf (DE); Clemens Daeschle, Walldorf (DE)

(73) Assignee: SAP AG, Walldorf (DE)

(21) Appl. No.: 14/068,523

(22) Filed: Oct. 31, 2013

(51) Int. Cl. G06F 9/445

(52) U.S. Cl. CPC G06F 9/44505
USPC 713/100

(57) ABSTRACT

In one embodiment, a method determines real-time information regarding changes to input data used to run an optimization. The optimization is run using a first computing system to generate a first optimization result within a first time window and the first computing system is configured to run the optimization periodically within subsequent time windows. The method determines when the changes to the input data indicate the optimization should be rerun. When the optimization should be rerun, the method causes a re-running of the optimization after the time window ends using a second computing system different from the first computing system. The re-running of the optimization using the changes and generating a second optimization result before a next time window for the first computing system to periodically run the optimization starts.

### BACKGROUND

Unless otherwise indicated herein, the approaches described in this section are not prior art to the claims in this application and are not admitted to be prior art by inclusion in this section.

Enterprise software systems use optimizers to perform optimization tasks. For example, labor-intensive industries may use the optimizers to optimize tasks, such as schedules for caring for patients at home, cleaning schedules, or schedules caring for patients in a hospital. For the example of caring for patients in their home, an optimization task may be who should go to which customer and when. A condition may be imposed on the optimization task to minimize the travel time, which may be a very difficult problem to solve and computationally expensive. Each night, the optimizer may calculate an optimization result that the optimizer needs to complete before the next morning for each employee. Once completed, the optimizer sends the optimization result to each employee, and each employee can then execute the plan.

The above process works if the input data to the optimizer does not change drastically in the real world. For example, unexpected events may occur while executing the plan, such as some employees may encounter traffic jams, some employees may not show up due to an illness, etc. As the number of unexpected events compound, the optimization result may become invalid or even counterproductive. Due to the large amount of time required to determine the optimization result, it may not be feasible to run the optimizer again to take into account the unexpected events. For example, the optimizer may run on limited computing resources that are designed to run overnight to finish the optimization by the morning. Running the optimization again once the day starts using the same computing resources would most likely take too long to provide a new plan to employees in time.

### SUMMARY

In one embodiment, a method determines real-time information regarding changes to input data used to run an optimization. The optimization is run using a first computing system to generate a first optimization result within a first time window and the first computing system is configured to run the optimization periodically within subsequent time windows. The method determines when the changes to the input data indicate the optimization should be rerun. When the optimization should be rerun, the method causes a re-running of the optimization after the time window ends using a second computing system different from the first computing system. The re-running of the optimization using the changes and generating a second optimization result before a next time window for the first computing system to periodically run the optimization starts.

In one embodiment, a non-transitory computer-readable storage medium contains instructions, that when executed, control a computer system to be configured for: determining real-time information regarding changes to input data used to run an optimization, the optimization being run using a first computing system to generate a first optimization result within a first time window and the first computing system configured to run the optimization periodically within subsequent time windows; determining when the changes to the input data indicate the optimization should be rerun; and when the optimization should be rerun, causing a re-running of the optimization after the time window ends using a second computing system different from the first computing system, the re-running of the optimization using the changes and generating a second optimization result before a next time window for the first computing system to periodically run the optimization starts.

In one embodiment, an apparatus includes: one or more computer processors; and a non-transitory computer-readable storage medium comprising instructions, that when executed, control the one or more computer processors to be configured for: determining real-time information regarding changes to input data used to run an optimization, the optimization being run using a first computing system to generate a first optimization result within a first time window and the first computing system configured to run the optimization periodically within subsequent time windows; quantifying an impact to the changes to the input data; determining when the impact violates a threshold; and when the impact violates the threshold, causing a re-running of the optimization after the first time window ends using a second computing system different from the first computing system and including more computing resources than the first computing system, the re-running of the optimization using the changes and generating a second optimization result before a next time window for the first computer system to periodically run the optimization starts.

The following detailed description and accompanying drawings provide a better understanding of the nature and advantages of particular embodiments.

### BRIEF DESCRIPTION OF THE DRAWINGS

FIG. 1 depicts a simplified system for performing real-time optimization according to one embodiment.

FIG. 2 depicts a more detailed example of running dedicated computer system 102 according to one embodiment.

FIG. 3 depicts a more detailed example of expandable computer system 106 according to one embodiment.

FIG. 4 depicts a flowchart of a method for real-time optimization according to one embodiment.

FIG. 5 depicts a more detailed example of a real-time optimization according to one embodiment.

FIG. 6 illustrates hardware of a special purpose computing machine configured with optimizers according to one embodiment.

### DETAILED DESCRIPTION

Described herein are techniques for an optimization system. In the following description, for purposes of explanation, numerous examples and specific details are set forth in order to provide a thorough understanding of particular embodiments. Particular embodiments as defined by the claims may include some or all of the features in these examples alone or in combination with other features described below, and may further include modifications and equivalents of the features and concepts described herein.

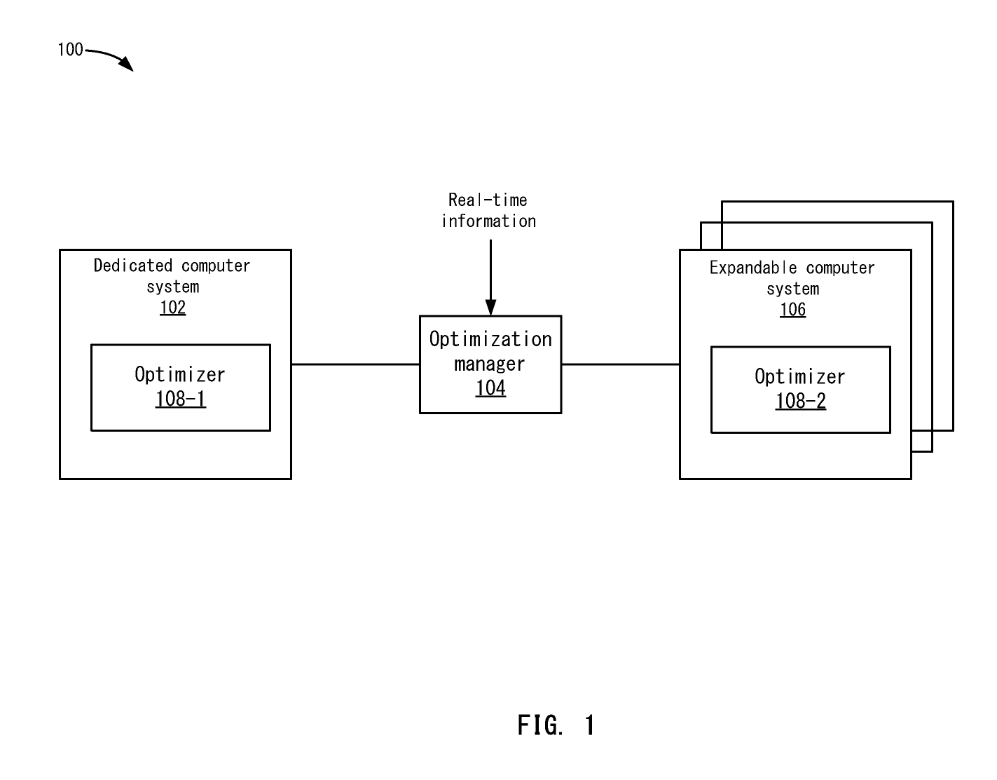

FIG. 1 depicts a simplified system 100 for performing real-time optimization according to one embodiment. System 100 includes a dedicated computer system 102, an optimization manager 104, and an expandable computer system 106. Dedicated computer system 102 includes an optimizer 108-1 and expandable computer system 106 includes an optimizer 108-2. Optimizer 108-1 and optimizer 108-2 may perform similar optimizations using a similar optimization algorithm.

Dedicated computer system 102 may be one or more physical machines that are dedicated to running the optimization periodically. For example, in one embodiment, a physical computing machine may include computing resources that run optimizer 108-1, which runs the optimization. In another embodiment, multiple physical computing machines include optimizer 108-1 and run the optimization in a distributive manner. Dedicated computer system 102 is dedicated to running the optimization periodically, such as in the example above, every night. In one embodiment, dedicated computer system 102 includes a first amount of computing resources that are not expandable dynamically. That is, additional computing resources may be added, but the computing resources cannot be added in time to re-run an optimization in real-time for the optimization result to be useful. Also, dedicated computer system 102 may be a fixed cost. For example, a company may own dedicated computer system 102.

Dedicated computer system 102 may be configured to run the optimization during a time interval that has a start time and an end time. For example, every day at midnight, dedicated computer system 102 may start the optimization using input data known at the time. Then, dedicated computer system 102 may be configured to process the input data to finish the optimization by substantially around 6:00 a.m. and output an optimization result. The dedicated computing resources have enough computing power to substantially finish the optimization in the time interval with variations in time appreciated. Also, in one embodiment, due to the computational requirements of the optimization, optimizer 108-1 may use most of or all of the computing resources available to dedicated computer system 102.

Optimization manager 104 may manage the optimization process. For example, optimization manager 104 may cause dedicated computer system 102 to run the optimization periodically. Optimization manager 104 may be included in a separate server in communication with dedicated computer system 102. Also, optimization manager 104 or parts of optimization manager 104 may be included in dedicated computer system 102 or expandable computer system 106.

As discussed above, real world information may affect the optimization result. For example, optimization manager 104 may receive real-time information, such as traffic information, information from employees, information from locations, such as hospitals, and other information that may affect an optimization result. As will be discussed in more detail below, optimization manager 104 may determine when changes to the input data that was initially used by optimizer 108-1 may require a new optimization to be run. In this case, optimization manager 104 may communicate with expandable computer system 106 to run the new optimization.

Expandable computer system 106 may include multiple computing machines in which optimizer 108-2 may run in an expandable fashion. For example, expandable computer system 106 may be in the “cloud” where a company can purchase an expandable amount of computing resources in the cloud from a cloud provider (e.g., a separate company) owns the computing resources in the cloud. Using the cloud, the company may dynamically increase the amount of computing resources available to optimizer 108-2 to rerun the optimization. In one example, the company may expand expandable computer system 106 from five to ten computing machines to run a new optimization result in real-time. Dynamically expanding from five to ten computing machines may make it possible for expandable computer system 106 to re-run the optimization where an optimization result can be useful. For example, expandable computer system 106 may be able to output a new optimization result in 30 minutes (instead of six hours with dedicated computer system 102), the new optimization result can then be sent to employees, who can change their tasks in time to be useful.

Because expandable computer system 106 is run in the cloud, a company running the optimization may be charged for the use of the computing resources. Thus, using more computing resources may cause the cost of calculating the optimization result to increase. Thus, in one embodiment, the cost for running an optimization result in expandable computer system 106 is higher than running the optimization in dedicated computer system 102. This may be because the company may have purchased dedicated computer system 102 and dedicated that system to running the optimization periodically. However, to run the optimization in expandable computer system 106, the company needs to reserve the space at the cloud provider. In some cases, the increased cost is worth running the optimization faster in expandable computer system 106. For example, the re-routing of employees due to traffic jams may outweigh the cost incurred for re-running the optimization.

In one embodiment, when optimization manager 104 receives the real-time information that changes the input data and optimization manager 104 decides that a new optimization result should be run, optimization manager 104 causes expandable computer system 106 to run the optimization using optimizer 108-2 instead of dedicated computer system 102. Optimizer 108-2 may run using a number of computing resources that are more than offered by dedicated computer system 102. This allows optimizer 108-2 to run the optimization faster. Once optimizer 108-2 runs the optimization, optimizer 108-2 may output the optimization where optimization manager 104 may send changes to the optimization result to users.

In one example, optimizer 108-1 runs all night and outputs optimization results in the morning. The optimization results detail where each employee should go to care for patients, and at what time. Optimization manager 104 checks the roads employees should take to reach patients for traffic jams. Also, if some employees do not show up in the morning, optimization manager 104 receives information for the absences. For example, a time check-in system may automatically determine which employees are absent or a user may enter the absences manually. Further, optimization manager 104 receives real-time data from hospitals if some patients need to be brought to the hospital and how long the patients will stay. Optimization manager 104 then evaluates the real-time information to determine if another optimization should be run in expandable computer system 106.

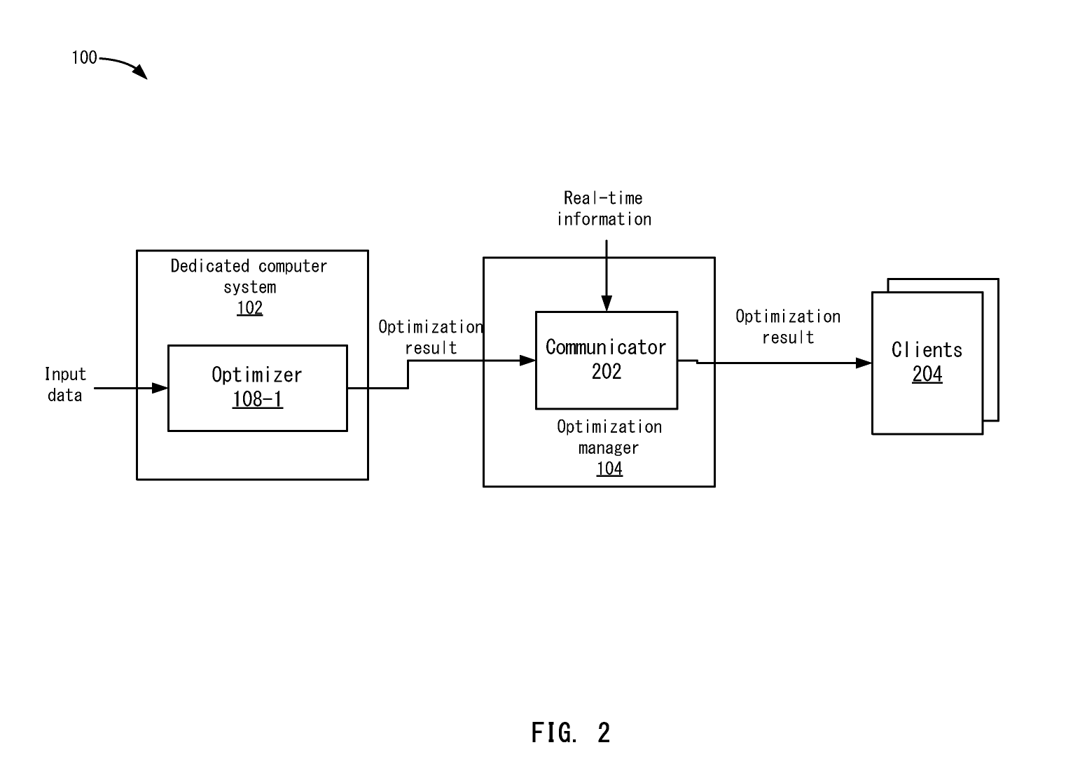

Before describing the evaluation in more detail, dedicated computer system 102 and expandable computer system 106 will be described. FIG. 2 depicts a more detailed example of running dedicated computer system 102 according to one embodiment. In dedicated computer system, optimizer 108-1 runs an optimization algorithm that outputs an optimization result based on the input data. As discussed above, optimizer 108-1 runs during a specified time interval to finish at a certain desired time.

Optimizer 108-1 may output the optimization result to optimization manager 104. A communicator 202 in optimization manager 104 may then communicate the optimization result or appropriate parts of the optimization result to clients 204. For example, communicator 202 may communicate to user devices, such as smartphones, a time and a location where a user is assigned. Communicator 202 communicates via any communication medium, such as e-mail, text message, telephone call, etc. In other embodiments, clients 204 may log into communicator 202 and request the optimization result. Although communicator 202 is described as being within optimization manager 104, communicator 202 may also be found in dedicated computer system 102.

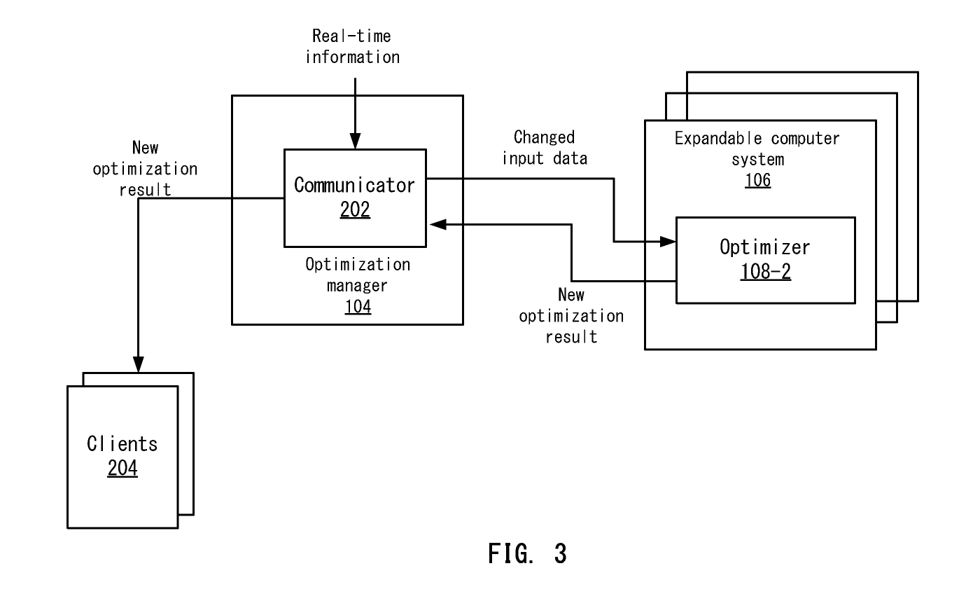

As discussed above, changes to the real-time information may cause optimization manager 104 to run the optimization again with expandable computer system 106. FIG. 3 depicts a more detailed example of expandable computer system 106 according to one embodiment. Optimizer 108-2 may run the optimization on an expandable number of computing resources in expandable computer system 106. Optimizer 108-2 receives the changes to the input data or a new input data set. Optimizer 108-2 then runs the optimization to determine a new optimization result. The new optimization result may be an entirely new optimization result. In other embodiments, the new optimization result may highlight the changes to the original optimization result. Optimizer 108-2 then outputs the new optimization result to communicator 202 of optimization manager 104. Communicator 202 may then communicate the new optimization result (or changes) to clients 204.

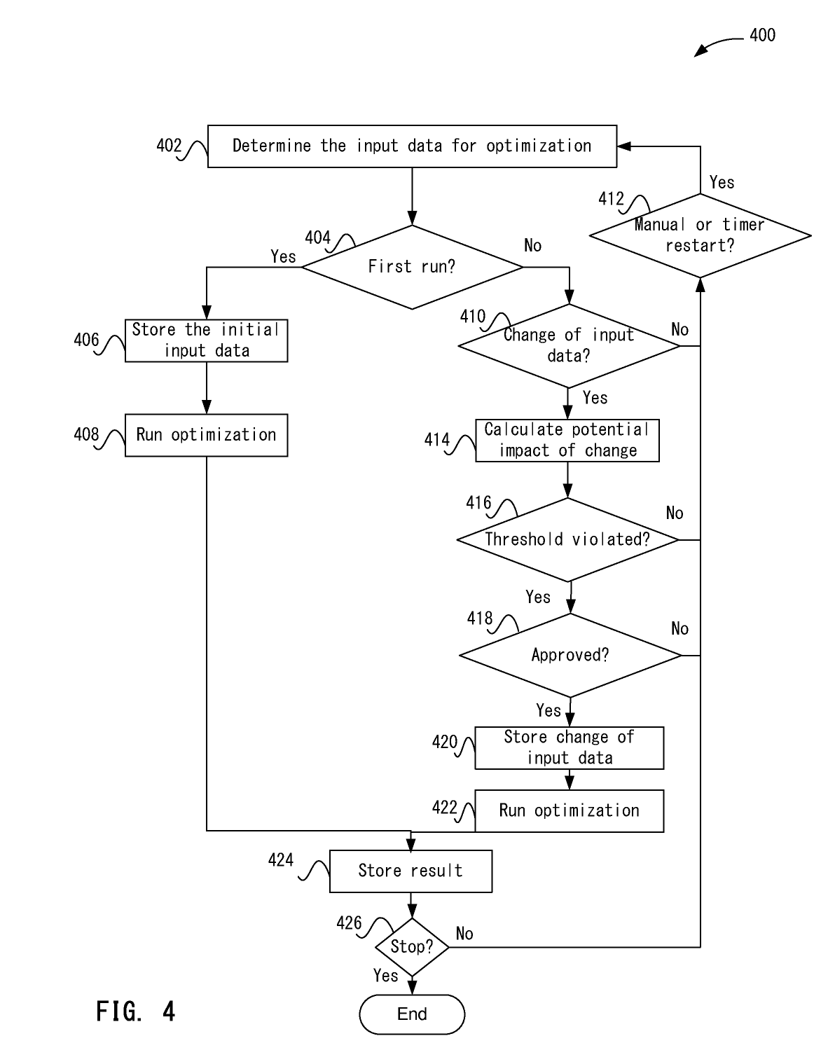

As mentioned above, optimization manager 104 may determine when real-time information warrants running a new optimization in real-time. Different methods may be performed to determine whether the optimization should be rerun. FIG. 4 depicts a flowchart 400 of a method for real-time optimization according to one embodiment. At 402, optimization manager 104 accesses the input data for the optimization. Optimization manager 104 may receive the input data from various sources, such as an enterprise software system (e.g., an enterprise resource planning (ERP) or supply chain management (SCM) system). At 404, optimization manager 104 determines if this is the first optimization calculation. If this is the first run, at 406, optimization manager 104 stores the initial input data. At 408, optimization manager 104 causes optimizer 108-1 to run the initial optimization based on the input data. Although optimization manager 104 is described as performing the determining of input data and the optimization, it will be understood that optimizer 108-1 may also determine the input data and run the optimization.

If this is not the first run, at 410, optimization manager 104 checks for real-time changes in the input data. For example, optimization manager 104 may receive real-time information from various sources, such as sensors, user devices, the Internet, etc. If there are no changes, the optimization may be later re-run based on a manual or timer based start at 412.

If there are changes, at 414, optimization manager 104 calculates the potential impact of the change. Optimization manager 104 may calculate the impact of the change based on various methods. For example, optimization manager 104 may look at pre-determined ranges of input data, a point system, or an evaluation of an increase in a variable, such as time/cost/difference. In one example, if the number of total drivers is not between a range of 4-6 drivers, optimization manager 104 calculates the impact as the number of drivers that are available, or the increase in time, in cost, or in distance, caused by the number of available drivers. At 416, optimization manager 104 determines if the impact violates a threshold. For example, if the number of available drivers is below four, then the impact of the changes violates the threshold. If not, the process reiterates to 406 where optimization manager 104 continues to check for changes in the input data.

If the impact violates the threshold, in one embodiment, optimization manager 104 may perform an extra check to determine if the rerunning of the optimization is warranted even though the impact violates the threshold. This check may be optional. For example, at 418, optimization manager 104 determines if the rerunning is approved. The approval may be automated or manual. In the manual approval, optimization manager 104 may output a message to a user to request approval for rerunning the optimization. The user may approve or deny the rerunning of the optimization, and optimization manager 104 receives a user input for the approval or denial. In the automatic approval, optimization manager 104 may weigh characteristics of the changed input data to determine whether or not to rerun the optimization. For example, optimization manager 104 may weigh the cost versus the impact. Optimization manager 104 determines the cost to run the optimization using expandable computer system 106 and also determines the impact the rerunning of the optimization. For example, there might be some deviations to the optimization results that are acceptable, such as only one road has a traffic jam that affects only one employee. Additionally, optimization manager 104 may assess a time limit to determine if the recalculation should be performed. For example, if the recalculation may take too long of a time to have an effect on the real world implementation of optimization result, then optimization manager 104 may not perform the recalculation. Also, when performing real-time optimizations, the approval should be automatic.

If optimization manager 104 determines the recalculation is not approved, the process reiterates to 412 where the optimization may be later re-run based on a manual or timer based start. However, if approved, at 420, optimization manager 104 stores the current input data, which includes the changes to the initial input data, and at 422, optimization manager 104 causes expandable computer system 106 to perform the optimization using the current input data. In one embodiment, optimization manager 104 calculates an amount of computing resources needed to rerun the optimization. The calculation may take into account the changes, a time limit in which the new optimization result is needed, and other factors. Optimizer 108-2 may then output the new optimization results. At 424, optimization manager 104 stores the optimization result for usage, such as for communication to clients 204, from either the first run or the re-running of the optimization.

At 426, optimization manager 104 determines if a stop flag is encountered indicating optimizations should be stopped. If the stop flag is not encountered, the optimization may be later re-run based on a manual or timer based start at 412. It should be noted if the timer based start has a very short timer, the optimization may be rerun in real-time.

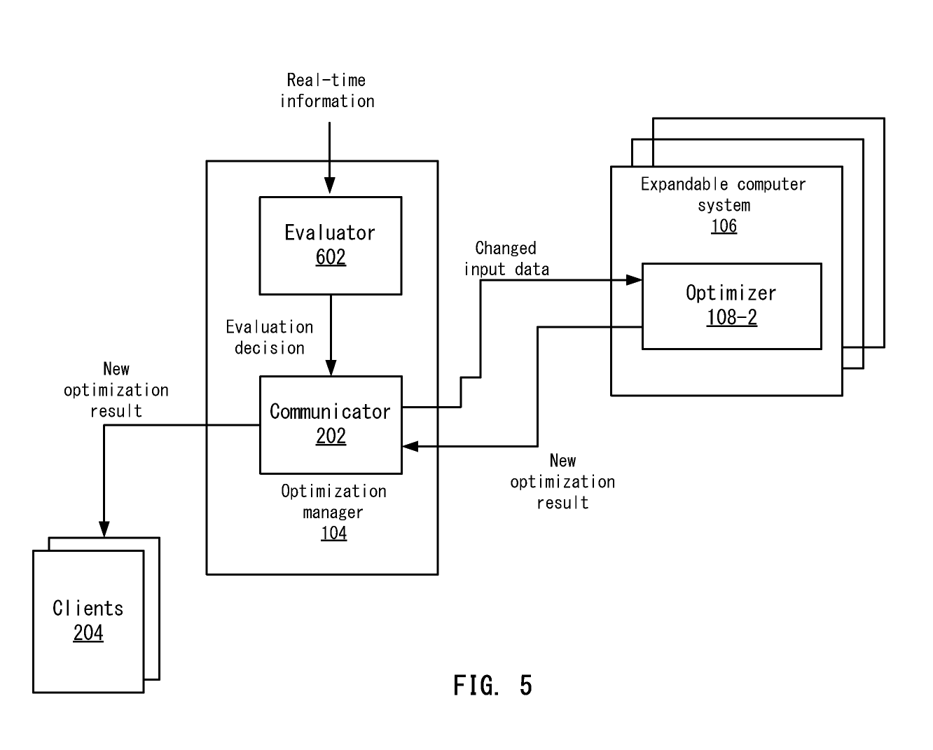

FIG. 5 depicts a more detailed example of real-time optimization according to one embodiment. An evaluator 602 of optimization manager 104 receives the real-time information. Evaluator 602 may then evaluate whether or not an optimization may be rerun as described above. Evaluator 602 outputs the decision to communicator 202, which then can cause the optimization to be run in expandable computer system 106. For example, communicator 202 requests a certain amount of computing resources in the cloud.

Expandable computer system 106 reruns the optimization using the changed input data. For example, optimizer 108-2 runs the optimization in the cloud and then outputs the new optimization result. Communicator 202 receives the new optimization result and can communicate the new optimization result to clients 204. For example, if any change in the optimization result affects a client/user, communicator 202 may send a changed optimization result to that client/user 204.

Accordingly, a company can perform real-time optimization that gets around the limit of running the optimization on the dedicated computer system the optimization typically runs on, but does not have sufficient computing resources to run the recalculation within a new time limit. Thus, the expandable computer system 106 is used, which may have massive parallelization and a large amount of computer resources available to do the new optimization in a quicker time period than dedicated computer system 102. Massive parallelization can be reached by e.g., running different optimization algorithms to solve the same problem in parallel, by using the same optimization algorithm in parallel with different initialization parameters, by massively parallelizing the algorithm itself or by using any combination of the methods. Also, if large data needs to be processed, expandable computer system 106 may use an in-memory database to speed up the process even more.

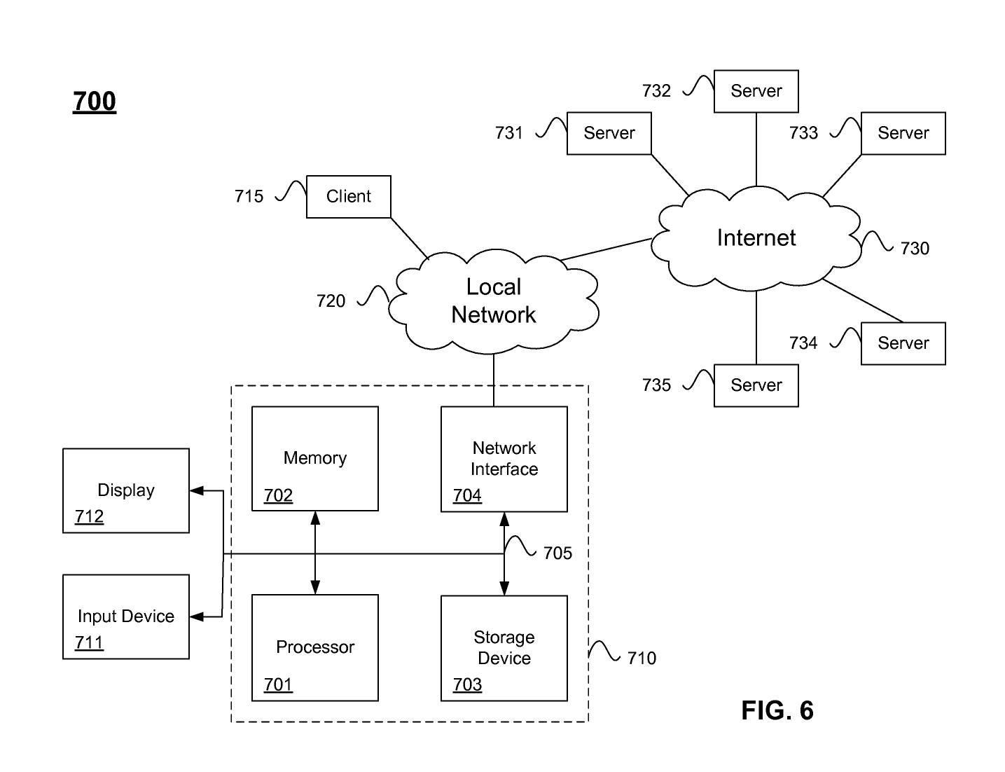

FIG. 6 illustrates hardware of a special purpose computing machine configured with optimizer 108-1 or 108-2 according to one embodiment. An example computer system 710 is illustrated in FIG. 7. Computer system 710 includes a bus 705 or other communication mechanism for communicating information, and a processor 701 coupled with bus 705 for processing information. Computer system 710 also includes a memory 702 coupled to bus 705 for storing information and instructions to be executed by processor 701, including information and instructions for performing the techniques described above, for example. This memory may also be used for storing variables or other intermediate information during execution of instructions to be executed by processor 701. Possible implementations of this memory may be, but are not limited to, random access memory (RAM), read only memory (ROM), or both. A storage device 703 is also provided for storing information and instructions. Common forms of storage devices include, for example, a hard drive, a magnetic disk, an optical disk, a CD-ROM, a DVD, a flash memory, a USB memory card, or any other medium from which a computer can read. Storage device 703 may include source code, binary code, or software files for performing the techniques above, for example. Storage device and memory are both examples of computer readable storage mediums.

Computer system 710 may be coupled via bus 705 to a display 712, such as a cathode ray tube (CRT) or liquid crystal display (LCD), for displaying information to a computer user. An input device 711 such as a keyboard and/or mouse is coupled to bus 705 for communicating information and command selections from the user to processor 701. The combination of these components allows the user to communicate with the system. In some systems, bus 705 may be divided into multiple specialized buses.

Computer system 710 also includes a network interface 704 coupled with bus 705. Network interface 704 may provide two-way data communication between computer system 710 and the local network 720. The network interface 704 may be a digital subscriber line (DSL) or a modem to provide data communication connection over a telephone line, for example. Another example of the network interface is a local area network (LAN) card to provide a data communication connection to a compatible LAN. Wireless links are another example. In any such implementation, network interface 704 sends and receives electrical, electromagnetic, or optical signals that carry digital data streams representing various types of information.

Computer system 710 can send and receive information through the network interface 704 across a local network 720, an Intranet, or the Internet 730. In the Internet example, software components or services may reside on multiple different computer systems 710 or servers 731-735 across the network. The processes described above may be implemented on one or more servers, for example. A server 731 may transmit actions or messages from one component, through Internet 730, local network 720, and network interface 704 to a component on computer system 710. The software components and processes described above may be implemented on any computer system and send and/or receive information across a network, for example.

Particular embodiments may be implemented in a non-transitory computer-readable storage medium for use by or in connection with the instruction execution system, apparatus, system, or machine. The computer-readable storage medium contains instructions for controlling a computer system to perform a method described by particular embodiments. The computer system may include one or more computing devices. The instructions, when executed by one or more computer processors, may be operable to perform that which is described in particular embodiments.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

The above description illustrates various embodiments along with examples of how aspects of particular embodiments may be implemented. The above examples and embodiments should not be deemed to be the only embodiments, and are presented to illustrate the flexibility and advantages of particular embodiments as defined by the following claims. Based on the above disclosure and the following claims, other arrangements, embodiments, implementations and equivalents may be employed without departing from the scope hereof as defined by the claims.

What is claimed is:

1. A method comprising:
determining, by a computing device, real-time information regarding changes to input data used to run an optimization, the optimization being run using a first computing system to generate a first optimization result within a first time window and the first computing system configured to run the optimization periodically within subsequent time windows;
determining, by the computing device, when the changes to the input data indicate the optimization should be rerun; and
when the optimization should be rerun, causing, by the computing device, a re-running of the optimization after the first time window ends using a second computing system different from the first computing system, the re-running of the optimization using the changes and generating a second optimization result before a next time window for the first computing system to periodically run the optimization starts.

2. The method of claim 1, wherein the second computing system is dynamically expandable by including more computing resources in the second computing system.

3. The method of claim 2, wherein the second computing system, when dynamically expanded, incurs an increased cost than running the optimization using the first computing system.

4. The method of claim 2, wherein the second computing system, when dynamically expanded, runs the optimization faster than the first computing system.

5. The method of claim 2, wherein the first computing system is not dynamically expandable.

6. The method of claim 1, wherein determining when the changes to the input data indicate the optimization should be rerun comprise:
quantifying an impact of the changes; and
determining when the impact violates a threshold; and
when the impact violates the threshold, causing the re-running of the optimization after the first time window ends.

7. The method of claim 6, further comprising:
when the impact violates the threshold, determining whether re-running of the optimization after the first time window ends is approved or disapproved;
when the rerunning is disapproved, not re-running of the optimization after the first time window ends; and
when the rerunning is approved, re-running of the optimization after the first time window ends.

8. The method of claim 1, further comprising communicating a portion of the second optimization result to one or more clients that received the first optimization result.

9. The method of claim 1, further comprising calculating an amount of computing resources needed in the second computing system to rerun the optimization based on the changes.

10. A non-transitory computer-readable storage medium containing instructions, that when executed, control a computer system to be configured for:
determining real-time information regarding changes to input data used to run an optimization, the optimization being run using a first computing system to generate a first optimization result within a first time window and the first computing system configured to run the optimization periodically within subsequent time windows;
determining when the changes to the input data indicate the optimization should be rerun; and
when the optimization should be rerun, causing a re-running of the optimization after the first time window ends using a second computing system different from the first computing system, the re-running of the optimization using the changes and generating a second optimization result before a next time window for the first computing system to periodically run the optimization starts.

11. The non-transitory computer-readable storage medium of claim 10, wherein the second computing system is dynamically expandable by including more computing resources in the second computing system.

12. The non-transitory computer-readable storage medium of claim 11, wherein the second computing system, when dynamically expanded, incurs an increased cost than running the optimization using the first computing system.

13. The non-transitory computer-readable storage medium of claim 11, wherein the second computing system, when dynamically expanded, runs the optimization faster than the first computing system.

14. The non-transitory computer-readable storage medium of claim 11, wherein the first computing system is not dynamically expandable.

15. The non-transitory computer-readable storage medium of claim 10, wherein determining when the changes to the input data indicate the optimization should be rerun comprise:
quantifying an impact of the changes; and
determining when the impact violates a threshold; and
when the impact violates the threshold, causing the re-running of the optimization after the first time window ends.

16. The non-transitory computer-readable storage medium of claim 10, further configured for:
when the impact violates the threshold, determining whether re-running of the optimization after the first time window ends is approved or disapproved;
when the rerunning is disapproved, not re-running of the optimization after the first time window ends; and
when the rerunning is approved, re-running of the optimization after the first time window ends.

17. The non-transitory computer-readable storage medium of claim 10, further configured for communicating a portion of the second optimization result to one or more clients that received the first optimization result.

18. The non-transitory computer-readable storage medium of claim 10, further configured for calculating an amount of computing resources needed in the second computing system to rerun the optimization based on the changes.

19. An apparatus comprising:
one or more computer processors; and
a non-transitory computer-readable storage medium comprising instructions, that when executed, control the one or more computer processors to be configured for:
determining real-time information regarding changes to input data used to run an optimization, the optimization being run using a first computing system to generate a first optimization result within a first time window and the first computing system configured to run the optimization periodically within subsequent time windows;
quantifying an impact to the changes to the input data;
determining when the impact violates a threshold; and
when the impact violates the threshold, causing a re-running of the optimization after the first time window ends using a second computing system different from the first computing system and including more computing resources than the first computing system, the re-running of the optimization using the changes and generating a second optimization result before a next time window for the first computer system to periodically run the optimization starts.

20. The apparatus of claim 19, wherein determining when the changes to the input data indicate the optimization should be rerun comprise:
when the impact violates the threshold, determining whether re-running of the optimization after the first time window ends is approved or disapproved;
when the rerunning is disapproved, not re-running of the optimization after the first time window ends; and
when the rerunning is approved, re-running of the optimization after the first time window ends.
